(19) United States
(12) Patent Application Publication
(10) Pub. No.: US 2024/0152666 A1
(43) Pub. Date: May 9, 2024

(54) SIMULATION OF ACCELEROMETER DATA

(71) Applicant: Hoffmann-La Roche Inc., Little Falls, NJ (US)

(72) Inventors: Jan Guy BOGAARTS, Basel (CH); Adrian DERUNGS, Basel (CH); Mattia ZANON, Basel (CH)

(21) Appl. No.: 18/277,763

(22) PCT Filed: Feb. 21, 2022
(86) PCT No.: PCT/EP2022/054287

§ 371 Date: Aug. 17, 2023

(30) Foreign Application Priority Data
Feb. 24, 2021 (EP) 21158872.8

(51) Int. Cl. G06F 30/20

(52) U.S. Cl. CPC G06F 30/20

(57) ABSTRACT

A computer-implemented method of simulating the effect of noise on an artificial accelerometer signal, the artificial accelerometer signal indicative of a body movement of an organism, includes the steps of: obtaining artificial acceleration data indicative of the body movement of the organism; obtaining noise characteristics of the accelerometer; and generating a simulated accelerometer signal based on the obtained artificial simulated acceleration data and obtained noise characteristics.

The present invention relates to computer-implemented methods for simulating the effect of noise on an artificial accelerometer signal, e.g. generated by a mobile device and movement features derived from artificial accelerometer signal. The artificial accelerometer signal may be indicative of a body movement of an organism.

### BACKGROUND TO THE INVENTION

Results of a two-minute walk test (TMWT) have been used as an outcome metric indicative of self-paced walking ability and functional capacity in health conditions such as chronic obstructive pulmonary disease, lower limb amputation, neuromuscular disease in adults/children, cardiac disease, functionally restrictive conditions like cystic fibrosis, and old age. For example, in cardiac disease, the results of the TMWT can be used for predicting peak oxygen update in patients with advanced heart failure.

The measurements of gait characteristics during a self-administered TMWT in persons with Multiple sclerosis (MS), using a single body-worn device, could provide high-density longitudinal information on disease progression, beyond what can currently be measured in a clinician-administered TMWT.

Neurological assessment such as this using smartphone apps, including gaming apps, for cognition and movement diseases such as Multiple sclerosis is influenced by the noise in the accelerometer embedded in the smartphone. In addition to this, there are variations in the accelerator noise which depend on the model of smartphone, which may be attributable to different manufacturing methods or different wear locations (on the organism).

### SUMMARY OF THE INVENTION

Broadly speaking, the present invention relates to the generation of a simulated accelerometer signal which takes into account the noise characteristics of a given accelerometer. In this way, it is possible to simulate a signal which would be generated by a specific accelerometer, e.g. on a mobile device as a result of a specific movement of an organism, for example walking. This simulated accelerometer data could also be obtained directly from an open source library or repository.

Specifically, a first aspect of the present invention provides a computer-implemented method of simulating the effect of noise on an artificial accelerometer signal, the artificial accelerometer signal indicative of a body movement of an organism, the method including the steps of: obtaining artificial acceleration data indicative of the body movement of the organism; obtaining noise characteristics of the accelerometer; and generating a simulated accelerometer signal based on the obtained artificial acceleration data and the obtained noise characteristics.

By generating a simulated accelerometer signal in this way, it is possible to evaluate the performance of a particular accelerometer at obtaining or generating data to be used in body movement analysis, such as gait analysis, without the need to take real accelerometer data, and to test that data. This is enabled by the use of artificial acceleration data, and the noise characteristics of a given accelerometer. In preferred cases the accelerometer is an accelerometer located on a mobile device, such as a smartphone.

Broadly speaking, the computer-implemented method of the first aspect of the invention may be split into three parts (i) obtaining artificial acceleration data; (ii) obtaining noise characteristics; and (iii) generating the simulated accelerometer data. We first discuss below the optional features pertaining to the first part of the invention.

The computer-implemented method of the first aspect of the invention requires that artificial acceleration data is obtained. In the present context, the term “artificial acceleration data” refers to data which is not directly measured by an accelerometer. For example, the artificial acceleration data may be simulated acceleration data. In some cases, the artificial acceleration data may be obtained from simulated position data. Specifically, obtaining artificial acceleration data may include: obtaining simulated position data, the simulated position data indicative of the changing position of a body part during the body movement of the organism; and calculating the artificial acceleration data from the obtained simulated position data. Here, it should be noted that term “body movement” should be understood to cover all musculoskeletal body movements, in particular those associated with walking. The body movement may be a cyclic, periodic, or quasi-periodic body movement, which may be referred to as a body movement cycle, such as a gait cycle. Alternatively, the body movement may be an irregular body movement.

The simulated position data may include a plurality of points, each point indicating the spatial-temporal position of a marker within a virtual environment in a duration of the body movement; and obtaining the simulated position data includes: attaching the marker to a musculoskeletal model in the virtual environment, the musculoskeletal model constructed to mimic the body movement of the organism; and tracking the position of the marker within the virtual environment throughout the duration of the body movement. The position is preferably measured relative to a global reference frame which is fixed relative to the virtual environment.

In implementations of the computer-implemented method which are employed to simulate the effect of accelerometer noise on acceleration data pertaining to an organism's gait, the musculoskeletal model is preferably a gait or walking model. This should be understood to be a computer model which may be simulated or based on real data, which represents the motion of an organism as it walks. The method may further include a step of obtaining the musculoskeletal model from a database, or other memory or repository. An example of a musculoskeletal model which may be used is described later in this application. The musculoskeletal model may be constructed to mimic the body movement of an organism with Multiple sclerosis, chronic obstructive pulmonary disease, a lower limb amputation, neuromuscular disease, or cystic fibrosis. The musculoskeletal model may be constructed to mimic the body movement of an organism at different ages, or with different stages of the conditions mentioned above.

Tracking the position of the marker within the virtual environment preferably includes measuring, detecting, determining, or otherwise identifying the spatial coordinates of the marker relative to the global reference frame, and storing the spatial coordinates in association with a temporal coordinate. In preferred cases, tracking the spatial-temporal position of the marker within the virtual environment includes sampling the spatial coordinates of the marker within the virtual environment at a sampling frequency. Alternatively put, tracking the spatial-temporal position of the marker within the virtual environment may include sampling the spatial coordinates of the marker within the virtual environment at regular sampling intervals. It will be understood that the sampling frequency is the inverse of the sampling interval. The provision of spatial-temporal position data enables the determination of the associated acceleration data. Specifically, calculating the artificial acceleration data may comprise calculating the acceleration of the marker within the virtual environment, i.e. in the global reference frame which is fixed relative to the virtual environment. Calculating the artificial acceleration data may comprise calculating or otherwise determining the second derivative of the spatial-temporal position data, with respect to time.

Calculation of the acceleration of the marker preferably includes a further step of applying a filter to the second derivative of the spatial-temporal position data, the filter configured to smooth the second derivative of the spatial-temporal position data of the marker. In some cases, calculation of the acceleration of the marker may include applying a single filter which both calculates and smooths the second derivative of the spatial-temporal position data in a single step. In preferred cases, the single filter may be a Kalman filter, or Kalman smoother. A more detailed explanation of the use of a Kalman smoother is set out later in this application. It is preferable that the acceleration data be smoothed, because it can then more accurately represent a natural, organic body movement, and take account of changes in motion which take place between the sampling intervals.

At this point, the acceleration data represents the acceleration of the marker within the global reference frame. However, in preferred cases, calculating the artificial acceleration data further includes transforming the calculated acceleration of the marker from the frame of the virtual environment to the local reference frame of the marker.

In order to generate artificial acceleration data as if measured by a sensor that moves and rotates with the body, it is necessary also to keep track of the orientation of the marker. In order to do this, the marker attached to the musculoskeletal model may be viewed as a primary marker, and obtaining the simulate position data may further include attaching a first secondary marker to the musculoskeletal gait model a first distance from the primary marker in a first direction, and tracking the position of the first secondary marker within the virtual environment; attaching a second secondary marker to the musculoskeletal gait model a second distance from the primary marker in a second direction, and tracking the position of the second secondary marker within the virtual environment; attaching a third secondary marker to the musculoskeletal gait model a third distance from the primary marker in a third direction, and tracking the position of the third secondary marker within the virtual environment, wherein the first secondary marker, the second secondary marker, and the third secondary marker are fixed relative to the primary marker. It is preferable that the first direction, the second direction, and the third direction are mutually orthogonal. Then, the imaginary lines connecting, respectively, the first, second and third secondary markers to the primary marker effectively define the x-, y-, and z-axes of the local reference frame of the primary marker. Preferably the first distance, the second distance, and the third distance are all a unit distance.

As with the primary marker, it is preferable that calculating the acceleration data further comprises calculating the acceleration of the first secondary marker, the second secondary marker, and the third secondary marker. This may be done in the same manner as for the primary marker.

Specifically, obtained simulated position data may include not only a plurality of points, each indicating the spatial-temporal position of the primary marker within the virtual environment (i.e. with respect to the global reference frame), but may also include: a plurality of points, each indicating the spatial-temporal position of the first secondary marker within the virtual environment in a duration of the body movement; a plurality of points, each indicating the spatial-temporal position of the second secondary marker within the virtual environment in a duration of the body movement; and a plurality of points, each indicating the spatial-temporal position of the third secondary marker within the virtual environment in a duration of the body movement.

Calculating the acceleration of the first secondary marker may comprise calculating or otherwise determining the second derivative of the spatial-temporal position data of the first secondary marker, with respect to time. Calculation of the acceleration of the first secondary marker preferably includes a further step of applying a filter to the second derivative of the spatial-temporal position data, the filter configured to smooth the second derivative of the spatial-temporal position data. In some cases, calculation of the acceleration of the marker may include applying a single filter which both calculates and smooths the second derivative of the spatial-temporal position data in a single step. In preferred cases, the single filter may be a Kalman filter, or Kalman smoother.

Calculating the acceleration of the second secondary marker may comprise calculating or otherwise determining the second derivative of the spatial-temporal position data of the second secondary marker, with respect to time. Calculation of the acceleration of the second secondary marker preferably includes a further step of applying a filter to the second derivative of the spatial-temporal position data, the filter configured to smooth the second derivative of the spatial-temporal position data. In some cases, calculation of the acceleration of the marker may include applying a single filter which both calculates and smooths the second derivative of the spatial-temporal position data in a single step. In preferred cases, the single filter may be a Kalman filter, or Kalman smoother.

Calculating the acceleration of the third secondary marker may comprise calculating or otherwise determining the second derivative of the spatial-temporal position data of the third secondary marker, with respect to time. Calculation of the acceleration of the third secondary marker preferably includes a further step of applying a filter to the second derivative of the spatial-temporal position data, the filter configured to smooth the second derivative of the spatial-temporal position data. In some cases, calculation of the acceleration of the marker may include applying a single filter which both calculates and smooths the second derivative of the spatial-temporal position data in a single step. In preferred cases, the single filter may be a Kalman filter, or Kalman smoother.

As again, it is possible to transform the acceleration calculated in respect of the secondary markers from the global reference frame to the local reference frame. Accordingly, calculating the artificial acceleration data may further include: transforming the calculated acceleration of the first secondary marker from the frame of the virtual environment to the local reference frame of the primary marker; transforming the calculated acceleration of the second secondary marker from the frame of the virtual environment to the local reference frame of the primary marker; transforming the calculated acceleration of the third secondary marker from the frame of the virtual environment to the local reference frame of the primary marker.

In all cases, the spatial-temporal position data, and acceleration data is preferably obtained for three orthogonal axes. It should also be noted that the velocity of the marker in question may also be calculated as an intermediate step. In order to identify the components of the position, velocity, or acceleration in the local reference frame, the vector indicating those quantities in the global reference frame may be projected on (dot- or inner-vector product) the respective unit vector representing the axis in the local reference frame. Specifically, the vector may be projected onto unit vectors connecting the primary marker, respectively, with the first, second, and third secondary markers (which define the axes of the local reference frame).

We now turn to optional features pertaining to the second part of the computer-implemented method of the first aspect of the invention, namely the obtaining of the noise characteristics of the accelerometer. In some cases, obtaining noise characteristics of the accelerometer may include obtaining the noise characteristics from a database. For example, if the accelerometer is located on a smartphone, the smartphone manufacturer may publish the relevant noise characteristics (see later), so that they can be straightforwardly obtained by practitioners of the computer-implemented method of the first aspect of the present invention. Alternatively, the noise characteristics may be measured or otherwise obtained from the accelerometer itself. Specifically, obtaining the noise characteristics of the accelerometer may include: recording a control acceleration signal from the accelerometer (for example, when it is stationary); and calculating the noise characteristics based on the control acceleration signal. In preferred cases, the noise characteristics include the Allan standard deviation σ_{Allan}

The Allan standard deviation σ_{Allan} is a standard deviation measure which depends on the sampling frequency with which the noise is sampled. More details regarding it are set out in the detailed description of the invention. Calculation of the noise characteristics based on the control acceleration signal may comprise calculating the Allan standard deviation σ_{Allan} of the control acceleration signal.

In some cases, the method may include a step of obtaining the Allan characteristics of the accelerometer, and deriving the Allan standard deviation σ_{Allan} for a given sampling frequency based on the Allan characteristics.

We now turn to optional features of the pertaining to the third step of the computer-implemented method of the first aspect of the invention.

Generating the simulated accelerometer signal may include generating a Gaussian white noise signal based on the noise characteristics of the accelerometer; and applying the Gaussian white noise signal to the simulated acceleration data. The Gaussian white noise signal is associated with a standard deviation σ, preferably determined based on the noise characteristics of the accelerometer.

Gaussian white noise is noise the amplitude of which has a Gaussian probability distribution. The probability density function p(z) of a Gaussian random variable z, in this case the amplitude of the noise is given by:

$$p ( z ) = \frac{1}{σ \sqrt{2 π}} {e}^{- \frac{{( z - μ )}^{2}}{2 {σ}^{2}}}$$

Here, σ is the standard deviation, and μ is the mean amplitude of the noise. In this case, the mean amplitude is preferably zero. When the mean amplitude of the noise is zero, the noise power is given by the variance σ² and the noise level, or amplitude (in acceleration units) is the standard deviation σ.

Specifically, the standard deviation σ is preferably determined based on a sampling frequency or sampling interval associated with the simulated acceleration data, and the noise characteristics of the accelerometer. The sampling frequency (or sampling interval) with which the spatial-temporal position data, and accordingly, the acceleration data are obtained, and the sampling frequency (or sampling interval) with which the noise characteristics are obtained may be different. In order to generate a signal which combines the two, it is necessary to scale the standard deviation of the noise characteristics based on the difference in sampling frequency/interval. Accordingly, the standard deviation σ of the Gaussian white noise signal may be calculated using the following relation:

$$σ = \frac{1}{\sqrt{Δ t}} {σ}_{Allan} = \sqrt{f} \times {σ}_{Allan}$$

Here, f and Δt are, respectively, the sampling frequency, and sampling interval associated with the sampling of the spatial-temporal data. σ_{Allan} represents the Allan standard deviation of the noise of the accelerometer for a sampling interval of one second, or a sampling frequency of one hertz. Preferably, the standard deviation σ is calculated for all three axes.

The computer-implemented method of the first aspect of the invention may further include additional steps which are performed after the generation of the simulated accelerometer signal. For example, the method may further include a step of extracting a parameter related to the body movement of the organism from the simulated accelerometer signal. It may be preferable to evaluate the effects of varying magnitudes of noise on the parameter extracted from the simulated accelerometer signal. Accordingly, the simulated accelerometer signal is a first simulated accelerometer signal, generated by applying a first Gaussian white noise signal the simulated acceleration data, the first Gaussian white noise signal having a first standard deviation σ₁, corresponding to an amplitude of σ₁; the parameter related to the body movement of the organism extracted from the first simulated accelerometer is a first body movement parameter and the method further includes: generating a second simulated accelerometer signal by applying a second Gaussian white noise signal the simulated acceleration data, the second Gaussian white noise signal having a second standard deviation σ₂, corresponding to an amplitude of σ₂; and extracting a second body movement parameter from the second simulated accelerometer signal. This may be repeated for any number of mean amplitudes.

The computer-implemented method of the first aspect of the invention may employ Monte Carlo techniques to derive a better understanding of the effects of accelerometer noise on, for example, the bias and variance of the values of the extracted parameters at different noise levels. In order to do so, for a given mean amplitude, a plurality of simulated accelerometer signals may be generated, a parameter extracted from each. These results may be plotted to give an indication of the bias and variance (or standard deviation) the values of that parameter as a result of noise of the given mean amplitude. Specifically, the method may further include: for a Gaussian white noise signal having a standard deviation σᵢ, corresponding to an amplitude σᵢ, generating a plurality of simulated accelerometer signals, and extracting a respective parameter from each of the plurality of generated simulated accelerometer signals. Preferably, the plurality of simulated accelerometer signals is no less than 20, more preferably no less than 50, and more preferably no less than 100. This process may be repeated for many different values of the mean amplitude σᵢ in order to determine the noise level at which point the noise starts to result in undesirable levels of bias or variance in the parameter. Results of Monte Carlo simulations along these lines are shown in the detailed description of this application.

One application of this invention is to establish the effect of noise on the results of two-minute walk tests (TMWTs). Accordingly, the extracted parameter may be a gait parameter. For example, the parameter may be step time, namely amount of time on average that each step takes. Alternatively, the parameter may be step power or step impulse. Calculation of these parameters is explained in the detailed description of the invention.

Before turning to a second aspect of the invention, we set out some possible technical applications of the simulated accelerometer signal. As we have discussed, a high-level aim of the invention is to establish the effects of noise on the results of motion-based tests for predicting or other digital tests of neurological impairment—in particular gait tests such as TMWTs. The outcome of such tests is some kind of classification, for example a classification of a stage or severity of a disease or condition affecting a patient's motor control. An important example of such a disease is Multiple sclerosis, and a method of interpreting machine learning results for classifying Multiple sclerosis, based on accelerometer signals, may be found in Creagh et al. (2021)¹. Each such test may have associated therewith a score representing the classification accuracy of the test, such as macro accuracy test, macro F1-score (MF1) and Cohen's kappa statistic, see He et al. (2009)², and Cohen et al. (1960)³. A computer-implemented method of determining a classification accuracy score may comprise: generating a simulated accelerometer signal according to the computer-implemented method of the first aspect of the invention, the simulated accelerometer signal encoding simulated accelerometer data; applying an analytical model to the simulated accelerometer data to generate an output; determining a classification accuracy score based on at least the simulated accelerometer data and the output of the analytical model; and outputting the classification accuracy score. The output of the analytical model may be indicative of a state or severity of a disease, or may provide a gait parameter such as a step power, step intensity, or step frequency. ¹ Creagh, A. P., Lipsmeier, F., Lindemann, M. et al. Interpretable deep learning for the remote characterisation of ambulation in Multiple sclerosis using smartphones. Sci Rep 11, 14301 (2021).² He, H. & Garcia, E. A. Learning from imbalanced data. IEEE Trans. Knowledge Data Eng. 21, 1263-1284 (2009).³ Cohen, J. A coefficient of agreement for nominal scales. Educ. Psychol. Measur. 20, 37-46 (1960).

The present invention enables simulation of accelerometer data with a varying range of noise levels. Accordingly, it may be used to determine the effect of varying noise levels on the classification accuracy of a given analytical model. For example, the computer-implemented method may comprise generating a first simulated accelerometer signal according to the computer-implemented method of the first aspect of the invention, the first simulated accelerometer signal encoding first simulated accelerometer data having a first noise level; applying an analytical model to the first simulated accelerometer data having the first noise level, to generate a first output; determining a first classification accuracy score based on at least the first simulated accelerometer data and the first output of the analytical model; and generating a second simulated accelerometer signal according to the computer-implemented method of the first aspect of the invention, the second simulated accelerometer signal encoding second simulated accelerometer data having a second noise level; applying the analytical model to the second simulated accelerometer data having the second noise level to generate a second output; determining a second classification accuracy score based on at least the second simulated accelerometer data and the second output of the analytical model; and outputting the first classification accuracy score and the second classification accuracy score.

This may, of course, be generalized for a plurality of different noise levels. Accordingly, the computer-implemented method may comprise: generating a plurality of simulated accelerometer signals according to the computer-implemented method of the first aspect of the invention, each simulated accelerometer signal encoding simulated accelerometer data having a respective noise level; applying an analytical model to each set of simulated accelerometer data having a respective noise level, each respective output of the analytical model indicative of a state or severity of a disease or condition; determining a respective plurality of classification accuracy scores, each based on respective simulated accelerometer data and the respective output of the analytical model; and outputting the respective plurality of classification accuracy scores.

In some cases it may be desirable to determine or approximate some kind of relationship between the noise level and the classification accuracy, based on the plurality of classification scores and the associated noise levels. Accordingly the computer-implemented method may comprise determining or approximating a relationship between noise level and classification accuracy score. The relationship may be defined in terms of one or more parameters. The relationship may be a linear relationship defined in terms of a gradient/slope and an intercept, but it will be appreciated that various different relationships may be defined.

The classification accuracy score for a given noise level or the relationship between the classification accuracy score and noise level, or other variation of classification accuracy score with noise may be used as an effect measure of the effectiveness of the analytical model. Accordingly, a computer-implemented method of selecting an analytical model to determine the state or severity of a disease or condition may comprise generating, for each of a plurality of analytical models, either: a classification accuracy score for a given noise level, or a relationship between a classification accuracy score and noise level (as outlined above); and selecting one of the plurality of analytical models based on the generated classification accuracy or relationship. Preferably, the analytical model with the greatest classification accuracy score would be selected, or the relationship which shows the least variation of classification accuracy score with noise. In the context of the present application, an “analytical model” is any mathematical model which may be applied to input data in order to determine clinically meaningful output data. The analytical model may be a machine-learning model, such as a deep learning model, which may be based, for example on artificial neural networks. The analytical model may be configured to determine a state or severity of Multiple sclerosis as outlined in Creagh et al. (2021) as cited earlier in this application.

Alternatively, rather than being used to determine the status or severity of a disease, the output of the analytical model may comprise a gait parameter. Gait parameters may be calculated as outlined in Bogaarts et al. (2021)⁴. ⁴ G. Bogaarts et al., “Simulating the impact of noise on gait features extracted from smartphone sensor-data for the remote assessment of movement disorders,” 2021 43rd Annual International Conference of the IEEE Engineering in Medicine & Biology Society (EMBC), 2021, pp. 6905-6910, doi: 10.1109/EMBC46164.2021.9630594.

In some cases, the analytical model may be configured to determine the gait parameter, and subsequently to determine a state or severity of a disease which affects a patient's motor control based on the gait parameter, such as Multiple sclerosis. The analytical model may be applied to one or more gait parameters derived from the results of a timed 25 foot walk test (T25FWT), a clinical test often used by neurologists to assess the walking ability of patients with Multiple sclerosis, see Rukina et al. (2021)⁵. ⁵ Rukina, D., Bogaarts, G., Dondelinger, F., Hubeaux, S., Zanon, M., Craveiro, L., Model, F., Cutter, G., Comi, G. (2021). Novel Smartphone Sensor-Based Scores for Remote Measurement of Gait and Hand Function Impairment in People with MS. Multiple Sclerosis. 27(2_suppl)

Alternatively, two separate analytical models may be used. In any event, the same process as outlined above, involving the calculation of classification accuracy scores and relationships between the classification accuracy scores and noise levels, may also apply to these analytical models. In some cases, the classification accuracy score (where classification gives rise to a discrete output), the score may indicate how well the analytical model predicts the value of a real-life parameter. Again, it may be possible to use these processes to determine a best-performing analytical model to investigate a particular aspect of a patient. In this manner, the computer-implemented method can use simulated accelerometer data in order to derive a clinically meaningful output in the form of a best-performing model which may then be used by a clinician to test for e.g. a particular condition based on real accelerometer data. The use of simulated data in this context is advantageous because it enables various methods to be tested without having large amounts of data available—clearly an advantage since the amount of “real” data available in this field is small.

A second aspect of the invention provides a computer program product comprising instructions which, when the program is executed by a computer, cause the computer to carry out the computer-implemented methods outlined above in respect of the first aspect of the invention. A third aspect of the invention provides a computer-readable storage medium having stored thereon the computer program product of the second aspect of the invention. A fourth aspect of the invention provides a system configured to perform the computer-implemented methods outlined above in respect of the first aspect of the invention. Accordingly, it will be noted that the optional features set out in this section relating to the first aspect of the invention apply equally well to the second, third, and fourth aspects of the invention, and therefore will not be repeated here, for reasons of conciseness.

### BRIEF DESCRIPTION OF THE DRAWINGS

The invention will now be described with reference to the drawings, in which:

FIG. 1 shows an example of a computer-implemented method of simulating the effect of noise on an artificial accelerometer signal.

FIG. 2 shows an example of how artificial acceleration data may be obtained.

FIG. 3A demonstrates how inverse kinematics can be used to generate a musculoskeletal model using experimental markers.

FIG. 3B shows a still of a walking model.

FIG. 4 shows a primary marker in place at the upper left thigh of a musculoskeletal model.

FIG. 5 shows three secondary markers in place in a musculoskeletal model.

FIGS. 6 and 7 show acceleration v. time plots from primary markers placed at the left thigh and lower back respectively.

FIG. 8 is an example of an Allan standard deviation plot.

FIG. 9 is an example of an Allan standard deviation plot obtained from an iPhone. There is deviation from the straight line as a result of increased uncertainty for the lower sampling frequencies.

FIGS. 10A to C are examples of simulated accelerometer signals at different noise levels.

FIGS. 11A and 11B show the results of a step detection algorithm on a simulated accelerometer signal with no noise, for a primary marker placed at the lower back, and left thigh respectively.

FIGS. 12A and 12B show the results of a Monte Carlo simulation at various noise levels for step power and step time, respectively.

FIGS. 13A and 13B show, respectively, how step power and step impulse can be calculated from acceleration data.

### DETAILED DESCRIPTION OF THE DRAWINGS

The invention will now be described with reference to drawings which explain a particular implementation of the invention and show results obtained using that implementation. It should be noted that this implementation is illustrative only, and should not be understood to restrict the scope of the claims.

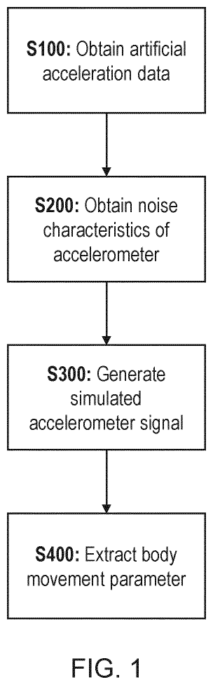

FIG. 1 shows a flow chart of the high-level computer-implemented method of the present invention. In step S100, artificial acceleration data is obtained; in step S200, noise characteristics of the accelerometer are obtained; in step S300, a simulated accelerometer signal is generated; and in step S400, a body movement parameter is extracted from the simulated accelerometer signal. It should be noted that steps S100 and S200 can be performed in either order.

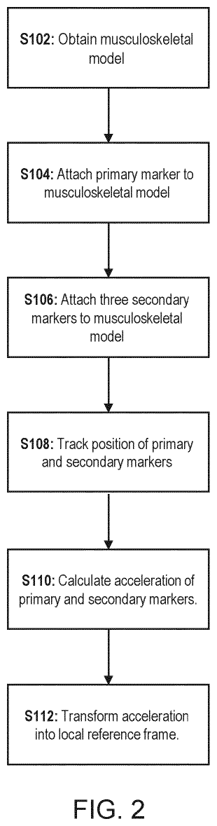

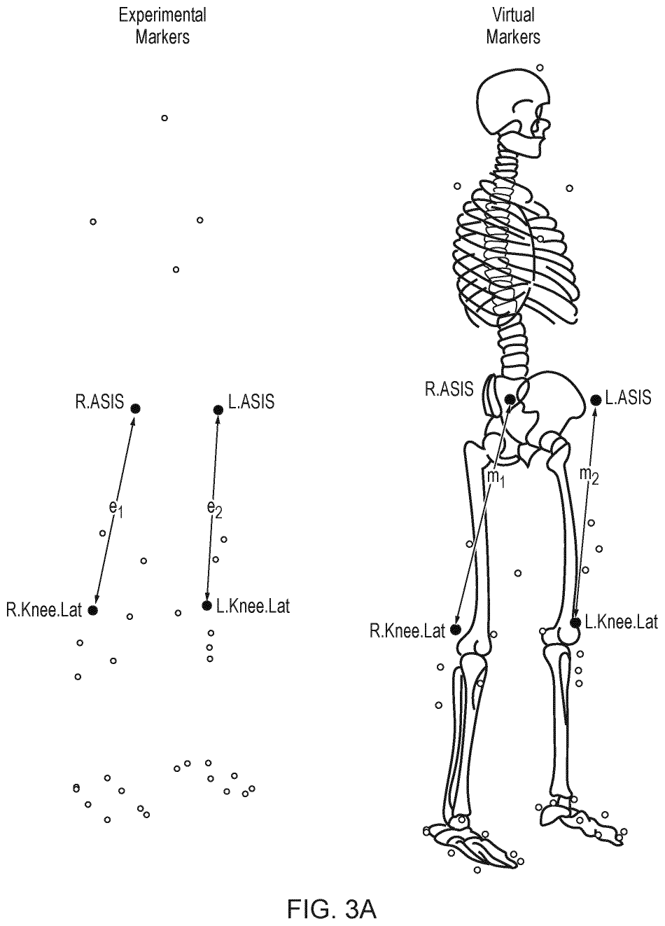

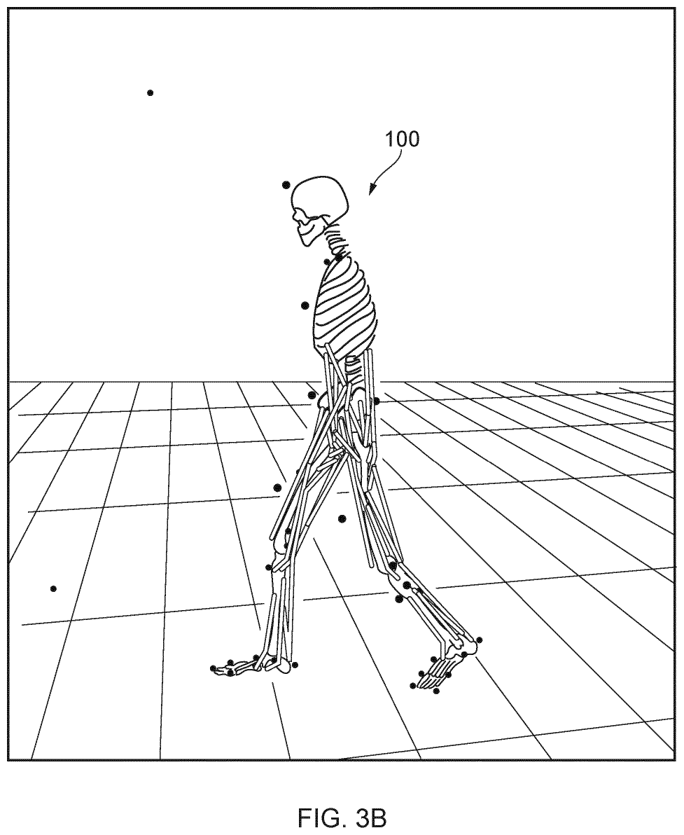

In step S100, artificial acceleration data is obtained. FIG. 2 shows another flow chart illustrating a series of steps by which the artificial acceleration data may be obtained. In step S102, a musculoskeletal model 100 is obtained. In one implementation, the musculoskeletal model 100 may be obtained from an open-source software package, such as OpenSim, which constructs musculoskeletal models using inverse kinematics, a process by which joint angles are estimated by matching experimental markers with virtual markers. This process is performed for each time point during a movement performed by a subject in a motion capture laboratory, and is illustrated in FIG. 3A. In the implementation described in this application, the Gait2354 model was used, which is a three dimensional model with 23 degrees of freedom of the human musculoskeletal model 100 with 54 musculotendon actuators. The motion data comes from a subject performing a single gait bout on a treadmill. After performing inverse kinematics, a walking model is generated that performs a single gait cycle in 1.2 seconds. A still of the walking model 100 is shown in FIG. 3B.

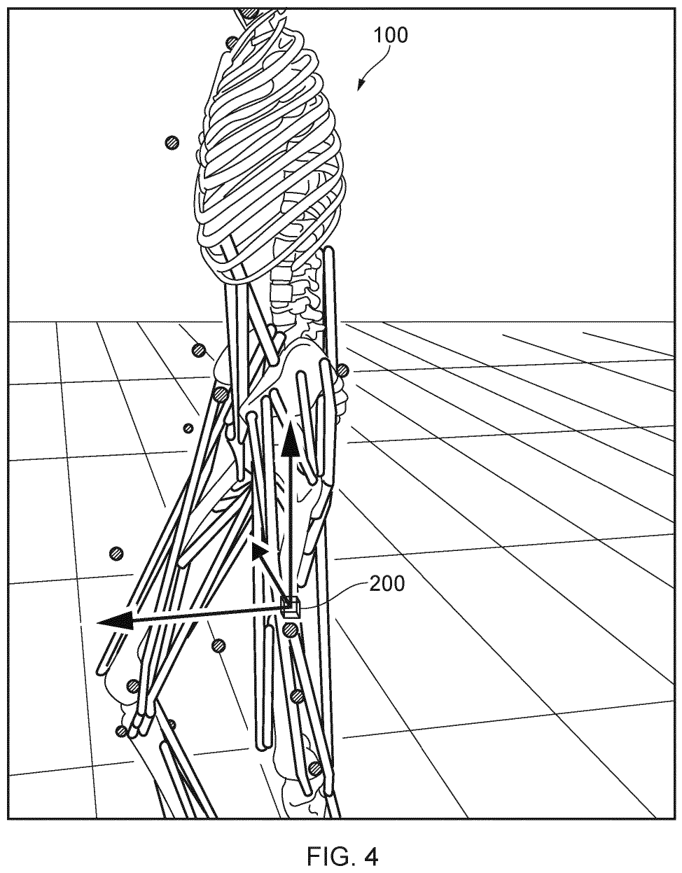

In step S104, a virtual (primary) marker 200 is attached to a segment of the musculoskeletal model 100. For example, the primary marker 200 may be attached to the thigh or lower back to mimic a phone in the pocket of the trouser or running pouch, respectively. FIG. 4 shows the primary marker 200 attached to the left thigh of the musculoskeletal model 100. By tracking the location of the primary marker 200 during the gait cycle it is possible to obtain an estimate of the acceleration signal that an accelerometer would have measured by taking the second derivative. In the present implementation, it is assumed that the majority of forces experienced by the virtual accelerometer (denoted by the primary marker 200) are due to linear accelerations. Even though human motion mainly involves rotations of bones around joints, it is assumed here that angular velocities are sufficiently small such that centrifugal forces are negligible compared to the forces due to linear acceleration.

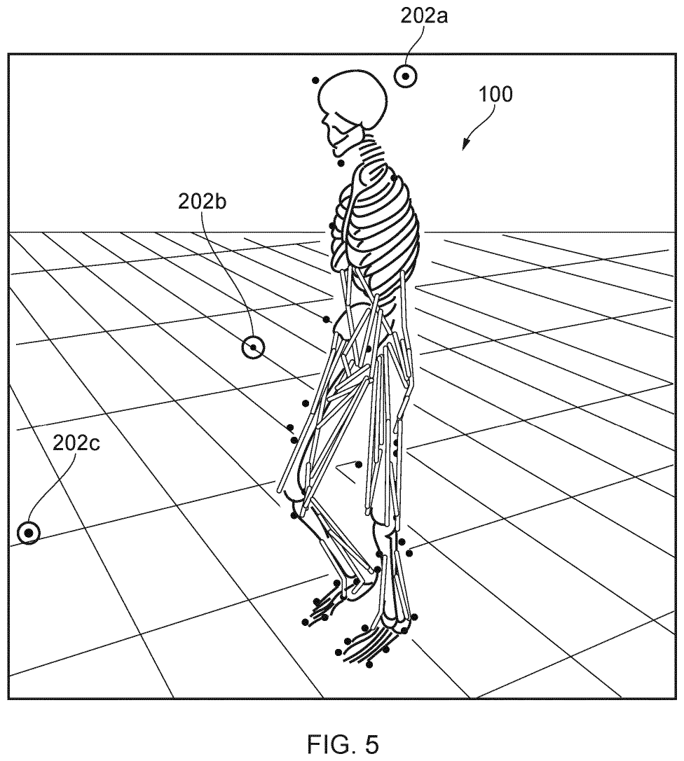

The position of the primary marker 200 is defined in the global reference frame. The global reference frame is attached to the environment and not to the moving model. To generate acceleration signals as if measured by a sensor that moves and rotates with the body, it is necessary to track of the orientation of the primary marker 200, too. To this end, in step S106, three secondary markers 202a, 202b, 202c are attached to the same model segment as the primary marker 200. Each of these three secondary markers 202a, 202b, 202c is placed one metre from the primary marker 200 in each of the three directions x, y and z. In this way they make-up a local reference frame that moves together with the primary marker 200. The musculoskeletal model 100, primary marker 200, and secondary markers 202a, 202b, 202c are shown in FIG. 5. Once the markers 200, 202a, 202b, 202c have been attached (virtually) to the musculoskeletal model 100, in step S108, the position of those markers 200, 202a, 202b, 202c are tracked over the course of a gait cycle, for example by sampling, at regular intervals, the spatial coordinates of the markers 200, 202a, 202b, 202c in the global reference frame of the virtual environment. This yields a total of 12 position time series in the global reference frame: position in each of the three dimensions, for four markers 200, 202a, 202b, 202c.

The acceleration of the primary marker 200, and the three secondary markers 202a, 202b,and 202c is then obtained in step S110. First, the acceleration of the markers 200, 202a, 202b, 202c is determined in the global reference frame, using a Kalman smoother. This is performed for each axis separately. An example process by which the acceleration data may be obtained is set out below, but it will be appreciated that other methods are possible.

The state of the virtual sensor is described by a position, velocity and acceleration:

$$[ \begin{matrix}x \\ \dot{x} \\ \ddot{x}\end{matrix} ]$$

State transitions are described by the transition matrix:

$$A = [ \begin{matrix}1 & Δ t & 0.5 Δ {t}^{2} \\ 0 & 1 & Δ t \\ 0 & 0 & 1\end{matrix} ] .$$

It is assumed that the sensor has constant acceleration that is subject to random variations resulting in the transition covariance vector:

$$q = [ \begin{matrix}\frac{Δ {t}^{3}}{6} \\ \frac{Δ {t}^{2}}{2} \\ Δ t\end{matrix} ] .$$

Hence, transitions are described by:

xₜ₌₁=A*xₜ₌₀+q*σ*q^{T}

Here, σ describes the assumed model noise. Since the data comes from a simulation a relatively high value for σ is selected. Because the model in the simulation has a net sideways movement a lower value of σ is selected for the z-axis such that this movement is smoothed out. In this way it is possible to avoid abrupt changes when gait bouts are concatenated. In preferred implementations of the method of the present invention, the value of σ is obtained automatically using an expectation-maximization process or algorithm.

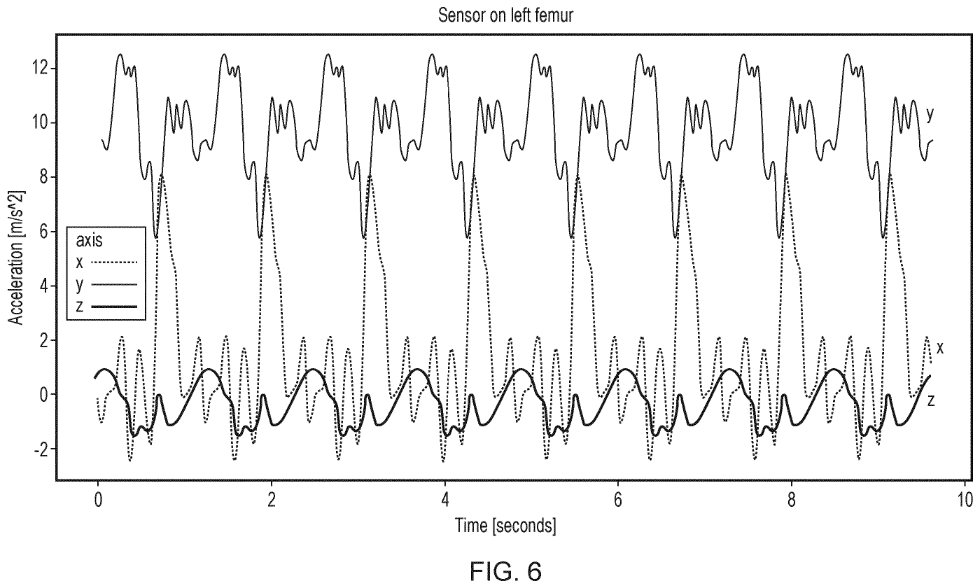

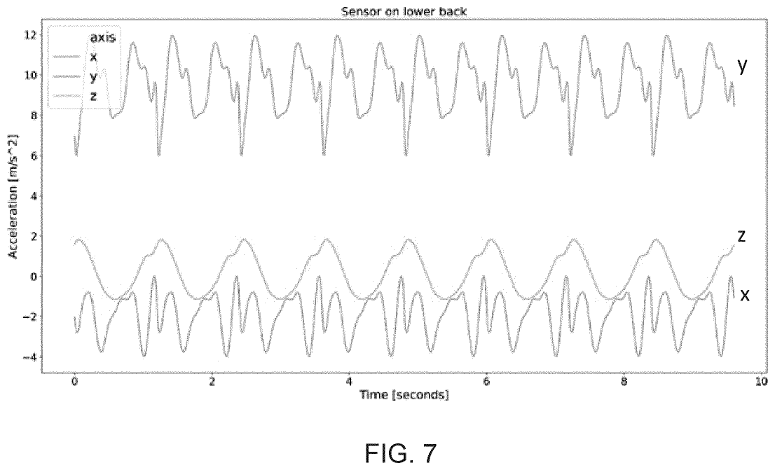

The above steps, forming step S110 of FIG. 2 result in acceleration in the global reference frame. Then, in step S112, the calculated acceleration signals are projected onto the axes of the local reference frame. This is done by taking the dot product between the acceleration vector and each of the three axes vectors (i.e. the vectors in the virtual environment which connect the primary marker 200, respectively to each of the secondary markers 202a, 202b, 202c). Before projection, the location of the primary marker is preferably subtracted from the locations of the axes vectors such that they originate from the origin of the local reference frame of the primary marker. FIGS. 6 and 7 show examples of the obtained acceleration signals for the left thigh and lower back respectively.

In step S200 of FIG. 1, noise characteristics of the simulated accelerometer are obtained. The noise characteristics can be obtained from a number of different smartphones. It is assumed herein that the only noise component which is large enough to have an impact is Gaussian white noise, since the tests are all relatively short. Characterization of white noise effectively means identifying the Allan standard deviation σ_{Allan} of an acceleration signal when the phone is stationary. Table 1 below shows the data from 5 different smart phones.

TABLE 1

| | Allan | Standard |
|---|---|---|
| | standard | deviation for |
| Model | deviation | OpenSim data |
| IPhone SE | 0.0005 | 0.0037 |
| IPhone XS | 0.0004 | 0.0031 |
| IPhone 11 | 0.0005 | 0.0039 |
| Samsung J7 | 0.0011 | 0.0086 |
| Samsung S7 | 0.0008 | 0.0065 |

The concept of Allan standard deviation σ_{Allan} is explained below.

Measurements taken by a sensor like the accelerometer in a smart phone experience perturbations as a result of noise that can be modelled as Gaussian white noise. This means that the standard deviation of the noise from the same sensor depends on the sampling frequency. If the sampling frequency decreases, the sampling time gets longer and the net effect of the Gaussian white noise is averaged out more. For very high sampling frequencies there is less time for the noise the average out and measurement noise will be higher. In order to address this, the Allan standard deviation σ_{Allan} was introduced, which is a log-log plot of the sampling interval against the measurement standard deviation.

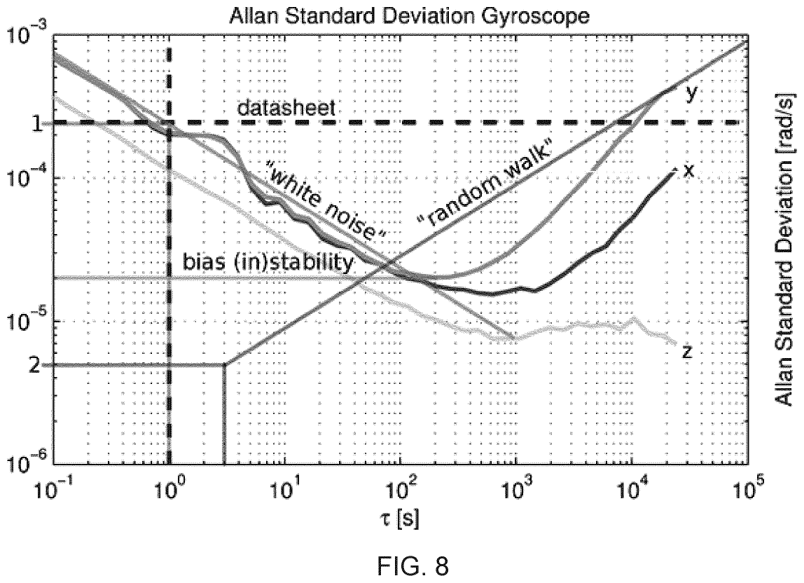

The Allan standard deviation σ_{Allan} is used to compare the noise of sensors with different sampling frequencies as well as to scale the standard deviation between different sampling frequencies. The latter function is used here when applying noise sampled at one sampling frequency to artificial acceleration data which was sampled at a different frequency. FIG. 8 shows an example of an Allan standard deviation σ_{Allan} plot where for high sampling frequencies a slope of −0.5 can be observed which is by definition Gaussian white noise. For lower sampling frequencies the noise increases again. The Allan standard deviation σ_{Allan} can also be expressed as a single number which is defined as the standard deviation for a sampling interval of 1 second. This σ_{Allan} is calculated for each of the 3 axes of each phone and scaled to match the sampling frequency of the OpenSim (i.e. the artificial acceleration) data using the following formula:

$$σ = \frac{1}{\sqrt{Δ t}} {σ}_{Allan} \qquad ( 1 )$$

Formula 1 is the result of fitting a line with a fixed slope of −0.5 through the Allan standard deviation σ_{Allan} plot and is derived as follows:

$$log log σ = Δ t + log log {σ}_{Allan} \\ log log σ = Δ {t}^{- 0.5} + log log {σ}_{Allan} \\ log log σ = \frac{1}{\sqrt{Δ t}} + log log {σ}_{Allan} \\ log log σ = \frac{1}{\sqrt{Δ t}} {σ}_{Allan} \\ σ = \frac{1}{\sqrt{Δ t}} {σ}_{Allan}$$

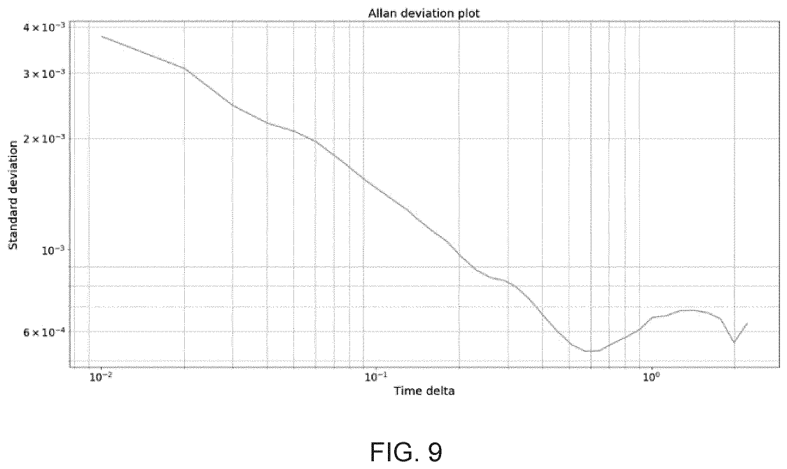

FIG. 9 shows an example of an actual Allan standard deviation plot of one of the iPhones. When the sampling interval increases less data is available to estimate the standard deviation and hence the uncertainty increases. For this reason weighted least squares are used when fitting the line where the weights scale with the inverse of the standard error of the standard deviation.

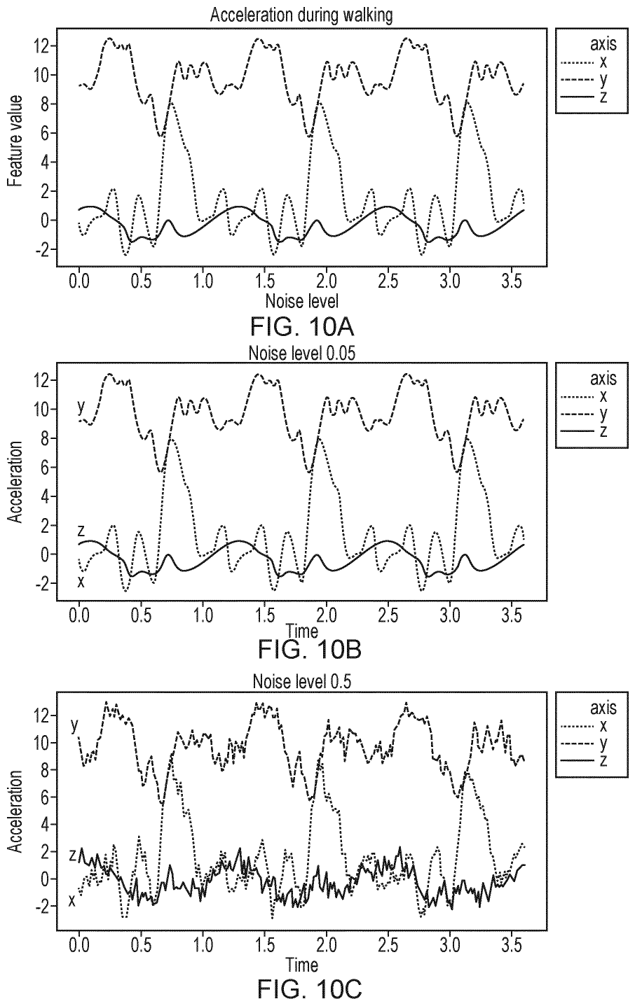

In step S300, the artificial acceleration data and the noise characteristics are used to generate the simulated accelerometer signal. Specifically, this may be done by concatenating the single-gait-bout time series to provide two minutes of data, and adding Gaussian white noise to each axis separately, to generate the simulated accelerometer data. This can repeated for different mean noise amplitudes. FIGS. 10A to 10C show examples of simulated accelerometer signals with different noise amplitudes (0, 0.05 ms⁻², 0.5 ms⁻²). The plot of FIG. 10B is representative of the noise levels in the tested smartphones.

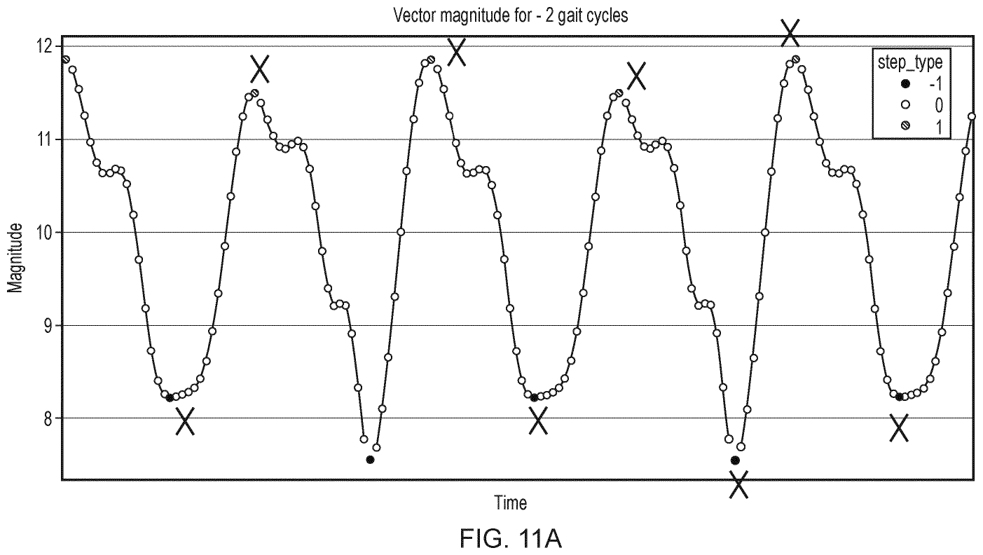

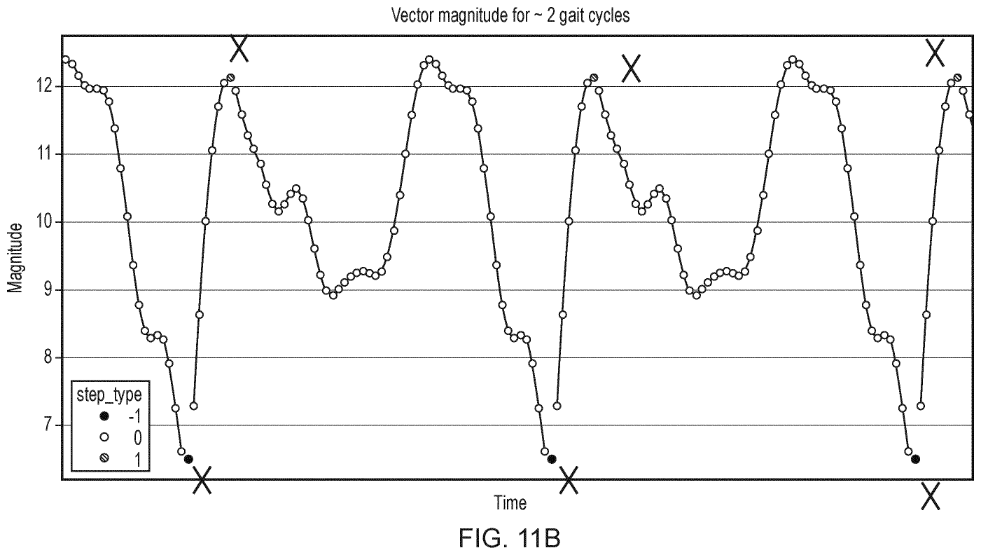

We now discuss the steps that may be carried out after the generation of the simulated accelerometer signal. In step S400, a parameter is extracted from the generated simulated accelerometer signal. For example a step detection algorithm, such as the one described in Lee et al. (2015)⁶, may be run on the simulated accelerometer data. This is important in the context of TMWTs, because most TMWT features depend on the accurate detection of steps. A step detection algorithm works by detecting peaks and troughs in the signal. For each gait cycle, the step detection algorithm searches for two peaks, and two troughs (one for each leg). FIGS. 11A and 11B show simulated accelerometer signals with no noise added, for a marker placed on the lower back of a musculoskeletal model, and a marker placed on the lift thigh of the musculoskeletal model, respectively. The crosses at the peaks and troughs illustrate the peaks and troughs actually detected by the step detection algorithm. It can be seen that for FIG. 11A, both peaks and troughs are detected. However, in FIG. 11B, it can be seen that only one peak and one trough is detected. In view of this, in the subsequent analysis, only the accelerometer data from FIG. 11A is used. ⁶ Lee, Hwan-hee, Suji Choi, and Myeong-jin Lee. “Step detection robust against the dynamics of smartphones.” Sensors 15.10 (2015): 27230-27250.

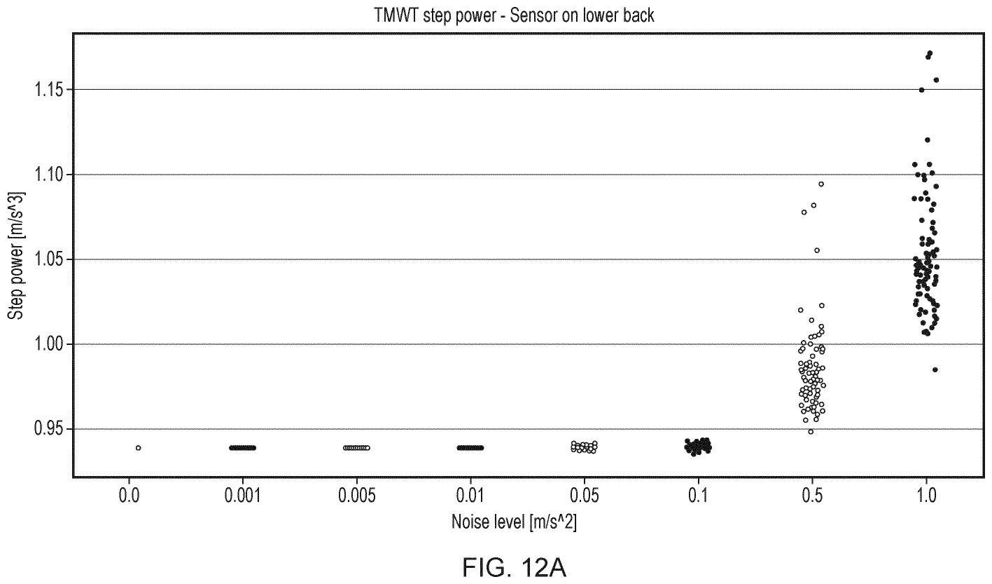

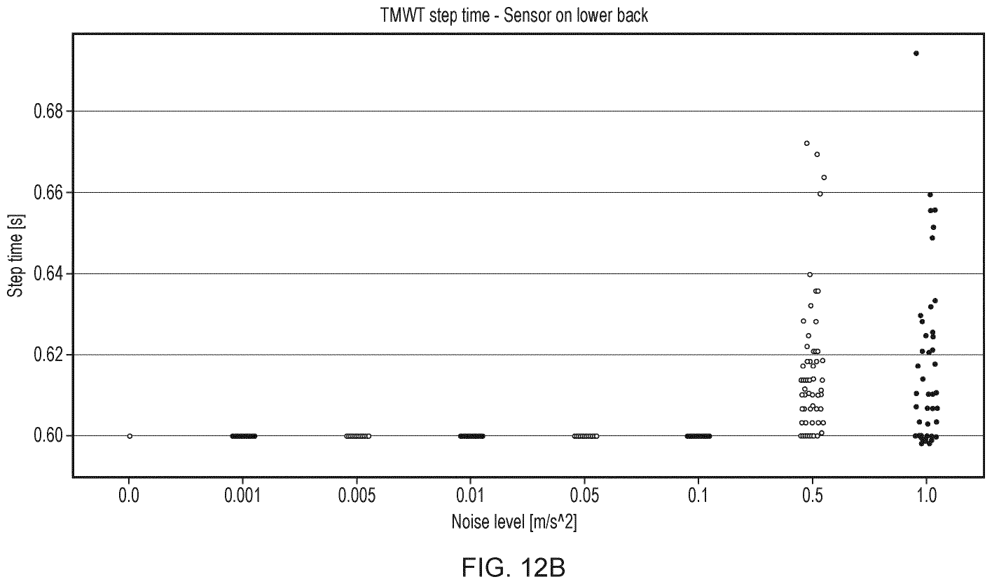

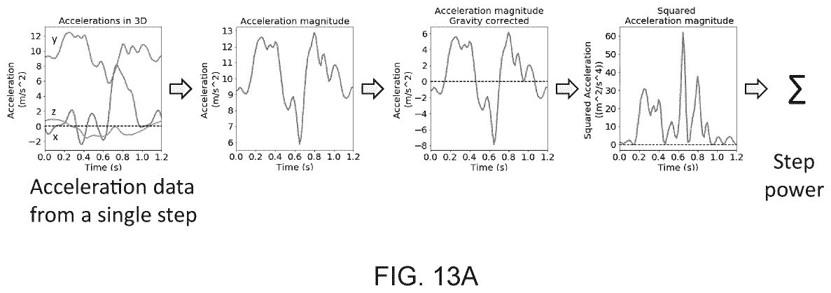

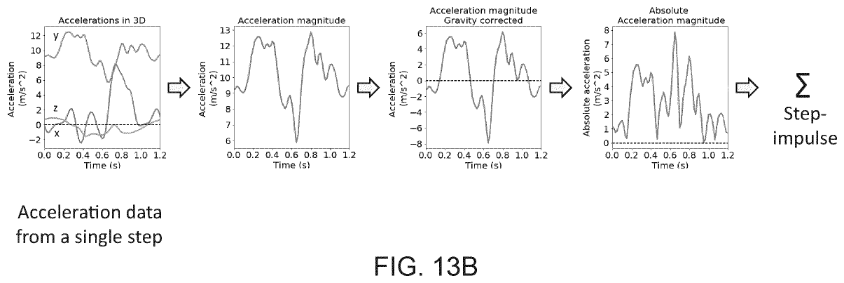

For each of a plurality of noise levels (0.0, 0.001, 0.005, 0.01, 0.05, 0.1, 0.5, 1.0 ms⁻²), 100 Monte Carlo repetitions were performed, to extract, from the simulated accelerometer signal of FIG. 11A, a step time, and step power parameter, the results shown in FIGS. 12A and 12B respectively. As can be seen, up to a noise level of 0.5 ms⁻², the gait features are relatively unaffected by the noise. However, for higher noise levels it can be seen that noise introduces a positive bias for both features. This can be explained by steps (peaks or valleys) not being detected resulting in longer step times and higher step power. In addition to a bias also an increase in variance over the simulated features can be observed. It can be concluded that the algorithm is robust to Gaussian white noise up to a certain level. Beyond this level noise results in both a bias as well as an increased uncertainty of the step time and step power. An alternative parameter, step impulse, may also be extracted from the simulated accelerometer signals. FIGS. 13A and 13B demonstrate what is meant by step power and step impulse.

1. A computer-implemented method of simulating an effect of noise on an artificial accelerometer signal, the artificial accelerometer signal indicative of a body movement of an organism, the method comprising:
obtaining artificial acceleration data indicative of the body movement of the organism;
obtaining noise characteristics of an accelerometer;
generating a simulated accelerometer signal based on the obtained artificial acceleration data and the obtained noise characteristics.

2. The method of claim 1, wherein:
obtaining artificial acceleration data includes:
obtaining simulated position data, the simulated position data indicative of a changing position of a feature during the body movement of the organism; and
calculating the artificial acceleration data from the obtained simulated position data.

3. The method of claim 2, wherein:
the simulated position data includes a plurality of points, each point indicating a spatial-temporal position of a marker within a virtual environment in a duration of the body movement; and
obtaining the simulated position data includes:
attaching the marker to a musculoskeletal model in the virtual environment, the musculoskeletal model constructed to mimic the body movement of the organism;
tracking the spatial-temporal position of the marker within the virtual environment, throughout the duration of the body movement.

4. The method of claim 3, wherein:
calculating the artificial acceleration data comprises calculating an acceleration of the marker within the virtual environment.

5. The method of claim 4, wherein:
calculating the acceleration of the marker within the virtual environment comprises calculating the second derivative with respect to time of the spatial-temporal position, and applying a filter to the simulated position data, the filter configured to smooth the second derivative of the spatial-temporal position.

6. The method of claim 5, wherein:
the marker attached to the musculoskeletal model is a primary marker; and
obtaining the simulated position data further includes:
attaching a first secondary marker to the musculoskeletal model a first distance from the primary marker in a first direction, and tracking the spatial-temporal position of the first secondary marker within the virtual environment;
attaching a second secondary marker to a musculoskeletal gait model a second distance from the primary marker in a second direction, and tracking the spatial-temporal position of the second secondary marker within the virtual environment;
attaching a third secondary marker to the musculoskeletal gait model a third distance from the primary marker in a third direction, and tracking the spatial-temporal position of the third secondary marker within the virtual environment,
wherein the first secondary marker, the second secondary marker, and the third secondary marker are fixed relative to the primary marker.

7. The method of claim 1, wherein:
obtaining noise characteristics of the accelerometer includes:
recording a control acceleration signal from the accelerometer; and
calculating the noise characteristics based on the control acceleration signal.

8. The method of claim 7, wherein:
calculating the noise characteristics based on the control acceleration signal comprises calculating the Allan standard deviation of the control acceleration signal.

9. The method of claim 1, wherein:
generating the simulated accelerometer signal includes:
generating a Gaussian white noise signal based on the noise characteristics of the accelerometer; and
applying the Gaussian white noise signal to the artificial acceleration data.

10. The method of claim 9, wherein:
the Gaussian white noise has a standard deviation, the value of the standard deviation being determined based on a sampling frequency or sampling period associated with the artificial acceleration data, and the noise characteristics of the accelerometer.

11. (canceled)

12. The method of claim 1, further comprising:
extracting a parameter related to the body movement of the organism from the simulated accelerometer signal.

13. The method of claim 12, wherein:
the simulated accelerometer signal is a first simulated accelerometer signal, generated by applying a first Gaussian white noise signal the artificial acceleration data, the first Gaussian white noise signal having a first standard deviation corresponding to a noise amplitude of the first standard deviation;
the parameter related to the body movement of the organism extracted from the first simulated accelerometer signal is a first body movement parameter and
the method further includes:
generating a second simulated artificial accelerometer signal by applying a second Gaussian white noise signal the artificial acceleration data, the second Gaussian white noise signal having a second standard deviation σ₂ corresponding to a noise amplitude of σ₂; and
extracting a second body movement parameter from the second simulated artificial accelerometer signal.

14. The method of claim 12, wherein:
the method includes, for a given mean amplitude σᵢ, of the Gaussian white noise, calculating a plurality of simulated accelerometer signals and extracting a respective parameter from each of the plurality of simulated accelerometer signals.

15. A computer-implemented method of determining a classification accuracy score, the method comprising:
obtaining artificial acceleration data indicative of a body movement of an organism;
obtaining noise characteristics of an accelerometer;
generating a simulated accelerometer signal based on the obtained artificial acceleration data and the obtained noise characteristics, the simulated accelerometer signal encoding simulated accelerometer data;
applying an analytical model to the simulated accelerometer data to generate an output;
determining a classification accuracy score based on at least the simulated accelerometer data and the output of the analytical model; and
outputting the classification accuracy score.

16. The method of claim 15, wherein the simulated accelerometer signal comprises a first simulated accelerometer signal encoding first simulated accelerometer data having a first noise level, the output comprises a first output, and the classification accuracy score comprises a first classification accuracy score, the method further comprising:
generating a second simulated accelerometer signal, the second simulated accelerometer signal encoding second simulated accelerometer data having a second noise level;
applying the analytical model to the second simulated accelerometer data having the second noise level to generate a second output; and
determining a second classification accuracy score based on at least the second simulated accelerometer data and the second output of the analytical model, wherein outputting the classification accuracy score comprises:
outputting the first classification accuracy score and the second classification accuracy score.

17. The method of claim 16, further comprising:
generating a plurality of simulated accelerometer signals, each simulated accelerometer signal encoding simulated accelerometer data having a respective noise level;
applying the analytical model to the encoded simulated accelerometer data associated with each of the plurality of simulated accelerometer signals to generate a respective plurality of outputs;
determining a respective plurality of classification accuracy scores, each based on respective simulated accelerometer data and the respective output of the analytical model; and
outputting the respective plurality of classification accuracy scores.

18. The method of claim 15, further comprising:
determining or approximating a relationship between noise level and classification accuracy score, wherein the relationship is defined in terms of one or more parameters.

19. (canceled)

20. The method of claim 15, further comprising:
generating, for each of a plurality of analytical models, either:
a classification accuracy score for a given noise level; or
a relationship between a classification accuracy score and noise level; and
selecting one of the plurality of analytical models based on the generated classification accuracy or relationship.

21. The method of claim 15, wherein:
the output of the analytical model is indicative of a status or severity of a disease or condition affecting a user's motor control; or
the output of the analytical model is indicative of one or more gait parameters, wherein the one or more gait parameters comprise: step power, step intensity, or step frequency.

22. (canceled)

23. A system for simulating an effect of noise on an artificial accelerometer signal, the artificial accelerometer signal indicative of a body movement of an organism, wherein the system comprises:
one or more processors configured to:
obtain artificial acceleration data indicative of the body movement of the organism;
obtain noise characteristics of the accelerometer; and
generate a simulated accelerometer signal based on the obtained artificial acceleration data and the obtained noise characteristics.
